(19) United States
(12) Patent Application Publication
(10) Pub. No.: US 2025/0209623 A1
(43) Pub. Date: Jun. 26, 2025

(54) Nutrition Digestive Efficacy Assistant and computer implemented algorithm thereof

(71) Applicants: N.V. Nutricia, Zoetermeer (NL); NUTRICIA EARLY LIFE NUTRITION (SHANGHAI) CO., LTD., Shanghai (CN)

(72) Inventors: Gregg Ward, Shanghai (CN); Mengjin Liu, Shanghai (CN); Bingzhi Guo, Shanghai (CN); Yi Jin, Shanghai (CN); Yixiao Zheng, Shanghai (CN); Jinhui Hu, Shanghai (CN); Agathe Camille Foussat, Singapore (SG); Jiahang Song, Shanghai (CN); Thomas Ludwig, Utrecht (NL)

(73) Assignees: N.V. Nutricia, Zoetermeer (NL); NUTRICIA EARLY LIFE NUTRITION (SHANGHAI) CO., LTD., Shanghai (CN)

(21) Appl. No.: 19/077,070

(22) Filed: Mar. 12, 2025

Related U.S. Application Data
(63) Continuation of application No. PCT/CN2023/119098, filed on Sep. 15, 2023.

(30) Foreign Application Priority Data
Sep. 16, 2022 (WO) PCT/CN2022/119270

(51) Int. Cl. G06T 7/00

(52) U.S. Cl. CPC G06T 7/0012; G06T 2207/20; G06T 2207/30004

(57) ABSTRACT

A computer-implemented method of analyzing excreta, comprising: obtaining visual information of excreta, providing the visual information to an artificial intelligence (AI) model; processing the visual information using the AI model to determine a plurality of scales of the excreta; outputting the plurality of scales of the excreta, receiving at least one input to adjust the plurality of scales of the excreta, and providing suggestion based on the adjusted plurality of scales of the excreta.

### FIELD OF THE INVENTION

The present invention relates to nutrition Digestive Efficacy Assistant, especially an excreta analyzing method, apparatus, and computer implemented algorithm thereof.

### BACKGROUND

In the healthcare field, for example, to care-receivers like people (e.g., an infant or a senior person) and other animals (e.g., dogs, cats, chickens, cows, sheep, goats, etc.), advances in technology allow helping caregivers with the task of tracking the developments of the care-receivers and quickly detecting certain anomalies.

More specifically, when it comes to the nutrition of a human infant, it is important to detect whether there are anomalies in the digestive system, or whether the infant's body is absorbing all the nutrients it needs to. For assessing the performance of the digestive system, analyzing the stool pattern is known to provide good insights. Scales to compare stool with a set of stool analysis scale scores help to classify the type of stool and to retrieve conclusions from there. Examples of such scales are the Bristol Stool Form Scale (BSS) and the Amsterdam Stool Scale. The BSS, which consists of seven images of different stool consistencies, allows assessment of stool consistency (scale 1 for hard lumps to scale 7 for watery stools), in an objective manner. The BSS may also be used to characterize the stool of infants and other young children.

For example, for babies, health care professionals (HCPs) usually ask the caretakers questions about the consistency of the stool of the infants, and these questions are difficult to answer for most parents. When parents are asked to keep a log of the stool consistency of their infants, it is difficult for them to identify the stool consistency and the associated stool analysis scale score that fits a stool of their kids.

It would be desirable to have a system where parents and caregivers could keep track of the stool pattern, i.e., stool consistency, frequency and colour, of their babies in real time. It would further be desirable, that irrespective of which caregiver (parent, grandparent, nanny or day care) is changing the diaper, or helping the child to use a potty or toilet chair, the stool pattern is tracked in an objective and consistent manner. It would also be desirable to have a system that, based on the observed stool patterns, provides an indication, either that everything is normal, which would provide ease of mind to the parents and caregivers, or that the infant's stool pattern is not behaving as expected and that is advisable to visit an HCP.

The above desirable scenarios apply to other types of caretakers and care-receivers as well, e.g., pet owners, veterinaries or farmers for cows, sheep, goat, chicken, etc. The animals may be also in another stage expect for the infant stage.

Nowadays, portable computing devices, e.g., smartphones, tablet computers or other portable devices with mobile applications (apps) can make normal life tasks easier for the users, which can also be applied to keeping track of stool patterns. Programs or apps are known which allow to introduce or to capture images of stool and manually select a score of a stool analysis scale that better suits the stool on the image. Also programs or apps are known which allow the use of colour recognition techniques to automatically detect the colour of stool.

However, the accuracy of the recognition needs to be improved. Furthermore, other excreta, e.g., urine, may be analyzed in addition to improve the health analysis results for given better heal suggestions.

### SUMMARY OF THE INVENTION

The present invention relates to an excreta analyzing method, apparatus, and computer implemented algorithm thereof.

The present invention is according to the claims.

### BRIEF DESCRIPTION OF THE DRAWINGS

The present invention will be discussed in more detail below, with reference to the attached drawings, in which:

FIG. 1 shows a computer-implemented method of analyzing excreta.

FIG. 2 shows an example of a user interface for adjusting the scales.

FIG. 3 shows an apparatus.

FIG. 4 shows a computer-implemented method of providing diet suggestions.

### DESCRIPTION OF EMBODIMENTS

Embodiments of the present disclosure will be described herein below with reference to the accompanying drawings. However, the embodiments of the present disclosure are not limited to the specific embodiments and should be construed as including all modifications, changes, equivalent devices and methods, and/or alternative embodiments of the present disclosure.

The terms “have,” “may have,” “include,” and “may include” as used herein indicate the presence of corresponding features (for example, elements such as numerical values, functions, operations, or parts), and do not preclude the presence of additional features.

The terms “A or B,” “at least one of A or/and B,” or “one or more of A or/and B” as used herein include all possible combinations of items enumerated with them. For example, “A or B,” “at least one of A and B,” or “at least one of A or B” means (1) including at least one A, (2) including at least one B, or (3) including both at least one A and at least one B.

The terms such as “first” and “second” as used herein may modify various elements regardless of an order and/or importance of the corresponding elements, and do not limit the corresponding elements. These terms may be used for the purpose of distinguishing one element from another element. For example, a first printing form and a second printing form may indicate different printing forms regardless of the order or importance. For example, a first element may be referred to as a second element without departing from the scope the present invention, and similarly, a second element may be referred to as a first element.

It will be understood that, when an element (for example, a first element) is “(operatively or communicatively) coupled with/to” or “connected to” another element (for example, a second element), the element may be directly coupled with/to another element, and there may be an intervening element (for example, a third element) between the element and another element. To the contrary, it will be understood that, when an element (for example, a first element) is “directly coupled with/to” or “directly connected to” another element (for example, a second element), there is no intervening element (for example, a third element) between the element and another element.

The expression “configured to (or set to)” as used herein may be used interchangeably with “suitable for,” “having the capacity to,” “designed to,” “adapted to,” “made to,” or “capable of” according to a context. The term “configured to (set to)” does not necessarily mean “specifically designed to” in a hardware level. Instead, the expression “apparatus configured to . . . ” may mean that the apparatus is “capable of . . . ” along with other devices or parts in a certain context.

The terms used in describing the various embodiments of the present disclosure are for the purpose of describing particular embodiments and are not intended to limit the present disclosure. As used herein, the singular forms are intended to include the plural forms as well, unless the context clearly indicates otherwise. All of the terms used herein including technical or scientific terms have the same meanings as those generally understood by an ordinary skilled person in the related art unless they are defined otherwise. The terms defined in a generally used dictionary should be interpreted as having the same or similar meanings as the contextual meanings of the relevant technology and should not be interpreted as having ideal or exaggerated meanings unless they are clearly defined herein. According to circumstances, even the terms defined in this disclosure should not be interpreted as excluding the embodiments of the present disclosure.

Excreta of animals (e.g., waste produced by the animal bodies) can reveal many indications to the health conditions of the animals, e.g., human infants or other animals. However, there is a need for an easy and accurate method for the excreta analysis. The present invention relates to an excreta analyzing method, apparatus, and computer implemented algorithm thereof.

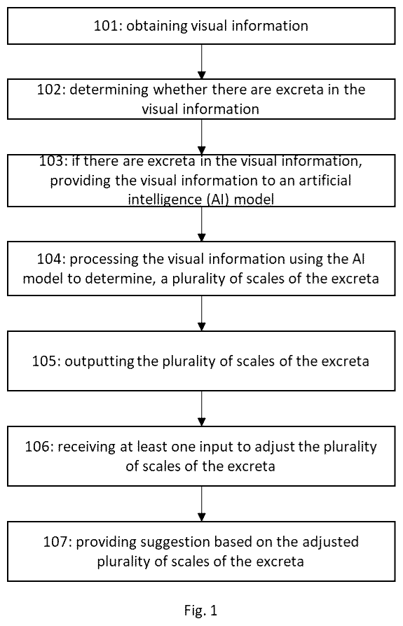

FIG. 1 shows a computer-implemented method of analyzing excreta. Some steps in FIG. 1 may be performed in a different sequence (e.g., step 107 may be performed after step 104 and/or again after step 106), may be merged (e.g., steps 101, 102 and 103), or may be omitted (e.g., steps 101, 102 or 105).

In step 101, visual information of excreta is obtained. The visual information may be at least one of the video information, image information, three-dimensional information and thermal image information. In the whole document, an image may be used as an example of the visual information, but it should be understood that the image is only an example and other forms of visual information are included in the present invention as well. In step 101, for example, an image or a video may be captured (either displayed or not displayed, may be stored in the memory or not), e.g., by a camera, thermal Imager, fetched from a memory, received from an external device via a telecommunication unit, or via other means. The visual information may have the excreta to be analyzed in it.

Excreta may be waste from an animal body, e.g., any one of a baby, an adult, a person, a dog, a cat, and other animals. The excreta may comprise at least one of urine and stool.

The visual information may be one of diaper visual information, nappy visual information, vessel visual information (e.g., bedpan, potty etc.), litter visual information, flushing toilet visual information, grass field visual information, ground visual information, etc, where excreta may be on or in.

In step 102, it may be determined whether there are excreta in the visual information. The determination may be by a pretrained artificial intelligence (AI) model, or by an image/visual information recognition algorithm.

If it is determined that there are no excreta in the visual information, then the method may stop. Or, an alert to the user may be outputted (via screen or speaker) to remind the user to capture or change the visual information, and then step 102 may be performed again with the new visual information. The determination step 102 may be omitted, e.g., it may be assumed that there are always excreta in the visual information.

In step 102, if it is determined that there are excreta in the visual information, the method may further determine the composition of the excreta, e.g., whether the excreta are only with stool or urine, or with both. This determination of the composition may be omitted.

In step 103, the method provides the visual information to an artificial intelligence (AI) model. The AI model may process the visual information and determine a plurality of scales of the excreta in step 104. The scales of the excreta may comprise color scales, consistency scales, volume scales, etc.

For example, if the excreta comprise stool, the plurality of scales of the excreta may comprise at least one of a color scale and a consistency scale of the stool. The color scales may include standard color scales or a subset of standard color scales. The consistency scales may include watery, soft, formed and hard based on the BSS or any other stool consistency standards.

If the excreta comprise urine, the plurality of scales of the excreta may comprise a color scale of the urine. The color scales may include standard color scales or a subset of standard color scales.

The AI model may be pretrained by training visual information (e.g., images), wherein the training visual information may be processed by a loss function. The loss function may comprise mixing two original visual information (e.g., images) together according to different transparency, generating mixing coefficients randomly for a plurality of times and letting the AI model learn according to a data distribution during the pre-training. Such a pre-training method can increase the prediction accuracy of the AI model. Furthermore, automated data augmentation may be performed to the training visual information before being used to train the AI model, such augmented training visual information can improve the prediction accuracy as well. The AI model may also be trained with a semi-supervised training method.

Before processing the visual information using the AI model in step 104, the visual information may be pre-processed, e.g., with at least one of flipping, lightening, darkening, and cropping. Such pre-processes can help the AI model to predict the scales more accurately.

In step 105, the determined plurality of scales of the excreta by the AI model is outputted, e.g., displayed or with sounds to indicate the scales. For example, the indication to a color scale may be text, colored image and/or colored text corresponding to the color scale. As examples, text “dark brown” may be displayed correspond to dark brown stool as determined by the AI model, or text “stool” with a dark brown color is displayed, or text “dark brown” with a dark brown color is displayed, or an overlying image with a dark brown color. Similarly, the indication to a consistency scale may be texts or an overlaying image indicating the consistency scale.

Augmented reality (AR) technologies may be used in the present invention. For example, when outputting the plurality of scales of the excreta, at least one virtual excreta overlaying object (e.g., a virtual excreta overlaying, an overlaying virtual stool image, virtual stool icon, a three-dimensional image, multimedia information, etc.) may be displayed, and a plurality of scales of the virtual excreta may be displayed according to the determined plurality of scales of the excreta. The virtual excreta (i.e., the virtual excreta overlaying object) may be displayed next to the excreta in the visual information according to the determined plurality of scales of the excreta.

For example, if the excreta in the visual information includes stool, an overlaying virtual stool object/icon (e.g., a stool shaped icon, a stool image with a predetermined trenchancy level, a copied image of the identified stool, or the combination thereof) may be displayed next to the captured visual information, e.g., next to the identified stool. In this example, the overlaying virtual stool object/icon may be displayed according to the determined plurality of scales of the stool, i.e., the plurality of scales of the virtual excreta may be displayed according to the determined plurality of scales of the excreta.

Taking color scale as an example, if the determined color scale of the stool in the visual information (e.g., image) is dark brown, the overlaying virtual stool object may be displayed in the corresponding dark brown color. For consistency scale, if the determined consistency of the stool is wet, the overlaying virtual stool object may indicate that the stool is wet, e.g., include a water drop icon in the overlaying virtual stool object, or with smooth stool surface in the overlaying virtual stool object.

The AR technology, especially with the virtual excreta overlaying object, may help the user (e.g., caregivers) to check the difference between scales of the excreta in the real visual information (i.e., the captured image that may be displayed) and the determined scales of the excreta by the AI model (via the virtual excreta overlaying object).

In step 106, at least one input may be received from a user to adjust the plurality of the scales of the excreta. This helps in improving the accuracy when determining the final/actual scales of the excreta. The inputs may be received via a touch screen with input receiving components displayed, or may be received with physical button inputs of a device, or any other means known to a person skilled in the art.

For example, if the virtual excreta are displayed, a user can easily see the difference between the color/consistency in the real visual information (i.e., obtained visual information) and the color/consistency in the virtual excreta. Then, the user can adjust the plurality of the scales of the excreta, wherein the virtual excreta may be displayed according to the change. The user may stop the input when there is almost no visible difference between the displayed virtual excreta and the real excreta in the obtained visual information.

Thus, when the received at least one input is configured to adjust the plurality of scales of the excreta, the plurality of scales of the virtual excreta may be displayed according to the adjusted plurality of scales of the excreta. In such a way, the predicted scales of the AI model can be further tuned by a user, such that the actual scales of the excreta can be accurately determined.

If the excreta comprise stool, the plurality of scales of the virtual excreta may comprise at least one of a color scale and a consistency scale of a virtual stool. If the excreta comprise urine, the plurality of scales of the virtual excreta may comprise a color scale of the virtual urine.

Similarly, if the excreta comprise stool, the adjusted plurality of scales of the excreta may comprise at least one of a color scale and a consistency scale of a virtual stool. If the excreta comprise urine, the adjusted plurality of scales of the excreta may comprise a color scale of the virtual urine.

Furthermore, since the adjusted plurality of scales of the excreta may be more accurate that the initially determined scales by the AI model, the adjusted plurality of scales of the excreta in step 105 may be used to re-train the AI model, such that the accuracy of the AI model may be further improved.

In step 107, suggestion based on the adjusted plurality of scales of the excreta (and/or some additional information, e.g., at least one of recorded diet information and at least one of gender, age, disease record, weight, and body length) is provided, e.g., displayed, or with sounds.

For example, the suggestion may comprise at least one health suggestion, which may comprise at least one of baby diet nutrient recommendation to improve baby stool function and health, at least one immunity system indicator of the baby and nutrition guidance to a breastfeeding mother.

The suggestion may comprise at least one meal plan, which may be further based on the dietary status and other additional information e.g., at least one of gender, age, disease record, weight, and body length, wherein the dietary status may be at least one of diet preference and allergen information of the care-receiver who produced the excreta.

The suggestion may further comprise a gut health score calculated based on the adjusted plurality of scales and/or the additional information.

The suggestion may further or only comprise comparable data of cases which have the same scales of the excreta as the adjusted plurality of scales of the excreta in the obtained visual information. For example, the adjusted plurality of scales of the excreta may indicate that the baby (who has produced the excreta) is in an abnormal/unhealthy state. Then, relevant data may be outputted on how often and/or what is the ratio of other babies having the same abnormal state. This may provide better indication to the caretakers (e.g., the parents) how urgent/severe the situation/condition of the baby is. If it is rather common, then the caretakers can relax, otherwise, they will know that they have to contact the doctors immediately.

Such suggestions may be generated according at least one looking-up table in a database cross-referenced to scales and/or the additional information. For example, a looking-up table may be specifically for four-month-old human infants who are breast-fed, and in this looking-up table, the possible values of the scales are all included and a combination of the values correspond to certain suggestions. E.g., dark brown and dry stool may lead to a suggestion to feed the infant additional amount water in every few hours; light-color urine may lead to a suggestion to feed less water to the infant. A meal plan may be included in the suggestion, e.g., certain recipes of meals from a database may be cross referenced with the scales (and/or the additional information) and suggested.

Such suggestions may be provided by a suggestion AI model, which may be pre-trained to provide suggestions according to the scales of excreta and/or the additional information, e.g., at least one of recorded diet information and at least one of gender, age, disease record, weight, and body length.

As an example, if the adjusted color scale indicates stool with a red, maroon, black, or pale color, an alert may be displayed to have the user's attention. Under such a condition, the suggestion may be further based on diet record by checking whether the diet record contains cause of the red, maroon, black, or pale color.

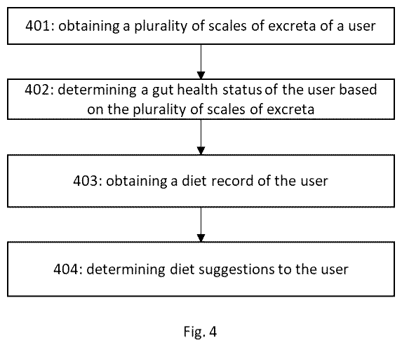

FIG. 4 shows a computer-implemented method of providing one or more diet suggestions (which may be performed by a device), for example, based on the plurality of scales of excreta of a user. FIG. 4 is combinable with the method in FIG. 1, for example, FIG. 4 may be considered as a more detailed method for step 107 in FIG. 1.

In step 401, a plurality of scales of excreta of a user may be obtained, which may be via the steps 101 to 105, or via steps 101 to 106 in FIG. 1, or may be inputted by a user directly to the device.

In step 402 (optional step), a gut health status of the user (who produces the excreta) may be determined based on the plurality of scales of the excreta. The determination may be further based on user information, for example, the age, geographic location, weight, gender, body mess index, diet record, etc. The gut health status may be provided with a score to indicate the health level of the gut development. In case the user is an infant (i.e., the infant as the user who produces the excreta), the gut health status may be the gut maturation of the infant, for example, indicating how well the gut of the infant is developed, which may be determined based on the plurality of scales of excreta and the user information of the infant. Step 402 may be optional and omitted in the method of FIG. 4, e.g., the method in FIG. 4 may only comprise steps 401 and 404, or steps 401, 403 and 404.

In step 403 (optional step), a diet record of the user may be obtained, e.g., from an input of a user of the device, or from a prestored database in the device, or from a server. The diet record may include the diet history information of the user (who produces the excreta) in a predetermined previous period, for example, diet information in the past week or three days. The diet information may include the diet time, food and drink amount, food and drink types, etc. This diet information may be used in step 402 as described above when determining the gut health status. Step 403 is optional and may be omitted in the method of FIG. 4, i.e., directly from step 402 (determining the gut health status) to step 404 (determining diet suggestions).

In step 404, one or more diet suggestions are determined, and/or outputted to the user via the device, which may be based on at least one of the plurality of scales of excreta, the gut health status, and the diet record. For example, if the excreta are with small balls, it may indicate constipation, then the suggestion may be to increase dietary fiber intake; If the stool is loose, it may suggest reducing dietary fiber or other foods that are easy to moisturize the intestines, such as bananas. Another example maybe, if the gut health status may indicate a premature development of the gut of an infant, the suggestions may comprise that the breast milk of the mother shall be the main diet of the infant for a period of future time and other supplementary food should be delayed until the gut of the infant is fully developed as expected. As an additional example, if the diet record shows that the user (who produce the excreta) has a habit of taking the same or similar diet (e.g., high fibre containing diet, high protein containing diet, etc.) in the past period, the suggestion may be to alternative the diet habit of the user.

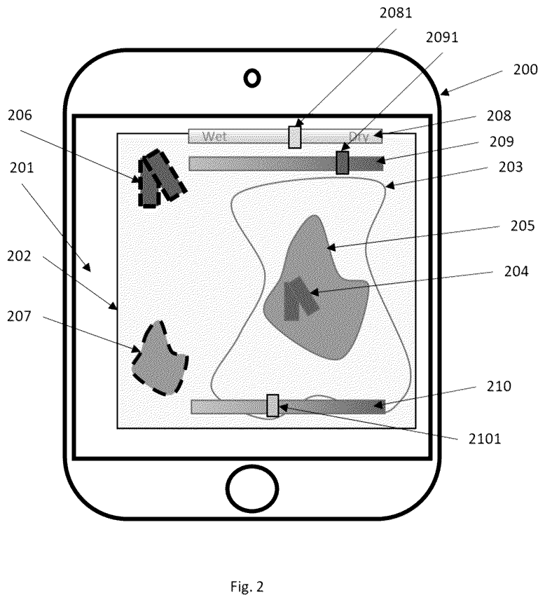

FIG. 2 is an example of a user interface for adjusting the scales of the excreta on an example apparatus 200.

The apparatus 200 may be a smart phone, a table let, a smart TV, a laptop, or any other computing devices. The apparatus 200 may comprise a screen 201, which may be a touch screen for both displaying and receiving input. The apparatus 200 may also comprise some physical input buttons.

In the example shown in FIG. 2, an image 202 (as an example of the obtained visual information) is shown on the screen 201, wherein a part of or the full image 202 is displayed. In the example image 202, there is a diaper 203. On the diaper 203, there are excreta (204 and 205). In this example, both stool 204 and urine 205 (or stain of urine) are on the diaper 203.

In steps 103 and 104, the image 202 may be processed by the AI model to determine the scales of the excreta. In this example, the determined scales of the excreta may be displayed in bar forms, e.g., with bars 208, 209 and 210, where the determined scale values are indicated by the indicators 2081, 2091, and 2101 on the bars 208, 209 and 210, respectively. Optionally, other forms of output may be used, e.g., text indications, color indications, turning wheel icons, arrows pointing to certain scales, etc.

In Step 106, inputs to adjust the scale values may be received. In order to better adjust the scales, virtual excreta overlying images 206 and 207 (also may be called excreta icons) are shown on screen 201. The stool icon 206 and the urine icon 207 may be with the same shapes as are in the diaper, or may be just representative icons (e.g., the stool icon is with a cartoon stool shape and the urine icon is with a water drop). The initial displayed colors of the excreta icons correspond to the determined excreta color scales by the AI model. For example, the initial color of the stool icon 206 may be the determined stool color scale of stool 204 on the image 202 by the AI model; similarly, the initial color of the urine icon 207 is the determined urine color scale of urine 205 on the image 202 by the AI model. The initial position of the indicators 2091 and 2101 of the bars 208 and 209 correspond to the initial color scales of the stool icon 206 and the urine icon 207, respectively. The stool 204 may also have a consistency scale which is determined by the AI model and indicated by the consistency bar 208 and indicator 2081, similarly as for the color scale.

Now a user can see the displayed colors of the stool icon 206 and the urine icon 207, and also the colors of the stool 204 and urine 205 in the image or even the stool and urine in the real-life diaper. After comparing them, the user may decide to adjust/correct the colors by moving the indicators 2091 and 2101 on the bars 209 and 210, respectively. When the indicators 2091 and 2101 are moved, the colors of the stool icon 206 and urine icon 207 may be changed according to the currently indicated color by the indicators 2091 and 2101, while the colors of the stool 204 and the urine 205 remain unchanged. The user may decide to stop changing the colors when the color differences between the stool icon 206 and the stool 204 (or the real stool) and between the urine icon 207 and the urine 205 (or the real urine) are minimized, i.e., the colors are almost the same (e.g., not able to be distinguished by bare eyes anymore), respectively. The adjusted/corrected color scales (and/or other scale information and/or the additional information) will be used to generate the suggestions in step 107 such that the suggestions are more accurate. Furthermore, the corrected colors may be used to train the AI model again, such that the future color predictions can be more accurate.

The consistency of the stool may be adjusted in a similar way as for the colors. In the example of FIG. 2, the stool consistency is indicated by bar 208 and the indicator 2081. The stool icon 206 itself may show cracks on it to indicate how dry the stool is (e.g., even loose stool pieces if it is indicated to be very dry). For example, the initial position of 2081 is according to the determined consistency scale by the AI model, and the stool icon 206 shows cracks accordingly to the determined scale. A user may compare the shown consistency of stool icon 206 and the stool 204 on the image 202 (or the real stool). If difference is identified by the user, inputs may be given to indicator 2081 to change the consistency of stool icon 206 while without changing the stool 204. Until the difference is minimized, the final consistency scale may be determined as the final adjusted consistency scale. The adjusted/corrected consistency scale (and/or other scale information and/or the additional information) will be used to generate the suggestions in step 107 such that the suggestion is more accurate. Furthermore, the corrected consistency scale may be used to train the AI model again, such that the future consistency prediction can be more accurate.

Alternatively, each element of the excreta (i.e., corresponding to each virtual excreta overlaying image/object), together with the corresponding scale adjustment user interface (e.g.,, the input receiving components like the bars including the indicators in FIG. 2), may be displayed once a time. In the example of FIG. 2, the stool icon 206 including its scale adjusting bars 208 and the urine icon 207 including its scale adjusting bar 2101 are displayed in the same screen display. Alternatively, in the example of FIG. 2, a first screen display may display the stool icon 206, the bars 208 and 209 (including the indicators 2081 and 2091), and the image 202; and a second screen display may display the urine icon 207, the bar 210 (including the indicator 2101) and the image 202.

Each of the virtual excreta overlying object/image together with the corresponding scale adjustment user interface may be displayed on the screen with a certain percentage of transparency. In this way, the background content on the screen (e.g., the image) will not be blocked entirely.

The virtual excreta overlaying objects may be omitted, e.g., only the input receiving components are displayed. For example, with the example in FIG. 2, the stool icon 206 and urine icon 207 may be omitted, and only the input receiving components are displayed, i.e., bars 208, 209 and 210 including the indicators 2081, 209a and 2101.

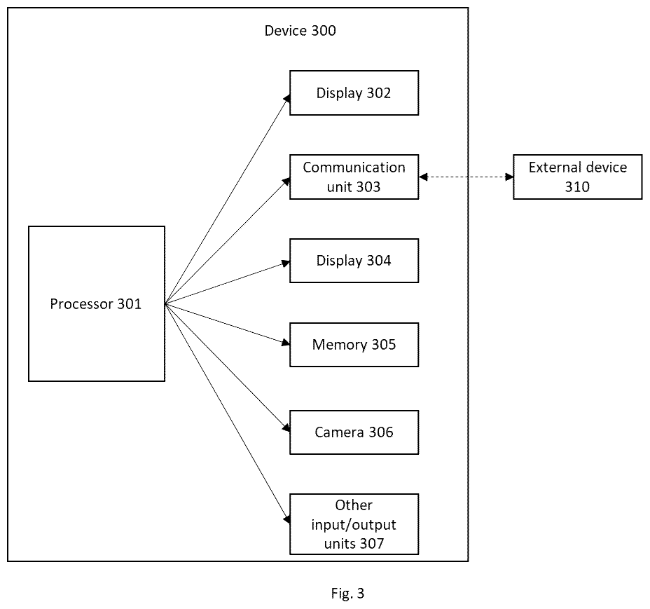

FIG. 3 shows a device 300 (e.g., the same as apparatus 200 in FIG. 2), e.g., a mobile phone, tablet, laptop, desktop, smart watch, a TV, etc., to perform the present invention.

The device 300 may comprise a processor 301, a display 302 (e.g., the same as the screen 201 in FIG. 2), a communication unit 303, a memory 305, a camera 306 and other input/output units 307.

The processor 301 is configured to perform the program/instructions (e.g., as in the methods of FIG. 1 and FIG. 4) stored in the memory 305, e.g., via controlling other components such as the display 302, the communication unit 303, the memory 305, the camera 306 and other input/output units 307.

The display 302 may be controlled by the processor 301 to perform the all the displaying function in the present invention such as in steps 105 and 107 and the example of displayed screen in FIG. 2. The display 302 may be a touch screen, which can receive input, for example in step 106 via the input receiving components displayed on the display 302 (e.g., in FIG. 2, the input receiving components including bars 208, 209 and 210 including the indicators 2081, 2091 and 2101).

The communication unit 303 may be controlled by the processor 301 to perform all communication function in the present invention. For example, if an external device 310 (e.g., an sever) is used to perform some functions in the steps of FIG. 1 (e.g., the visual information in step 101 may be obtained from the external device 310; steps 102 and 103 may be performed by device 300; steps 105 and 106 may be performed by device 300; the suggestion may be generated by the external device 310 and outputted by device 300. Or, all the steps may be performed on the user device 300; or a part of the steps is performed on the user device 300 and the remaining part of the steps is performed on at least one or more external devices 310), messages may be communicated via the communication unit 303. Optionally, the databases used in the present invention may be stored in the external device 310 or the user device 300, e.g., the look up tables for the suggestions.

The memory 305 may be configured to store the instructions and data to perform the methods of the present invention. For example, the look up tables for the suggestions and the obtained visual information may also be stored in the memory 305. The device 300 may provide at least one entry for the user to check/overviewing these data.

The camera 306 is configured to obtain visual information, e.g., capture images, as an example in step 101.

The other input/output units 307 may be configured to perform other input/output functions of the present invention, for example, to receive user input for adjusting the scales.

In the present invention, at least a part of the device (e.g., FIG. 3) or method (e.g., FIG. 1 or FIG. 4 a as computer implemented method) may be implemented as instructions stored in a non-transitory computer-readable storage medium, e.g., in the form of a program module, a piece of software, a mobile app, and/or other forms. The instructions, when executed by a processor (e.g., the processor 301), may enable the processor to carry out a corresponding function according to the present invention. The non-transitory computer-readable storage medium may be the memory 305.

A computer-implemented method of analyzing excreta, comprises: obtaining visual information of excreta; providing the visual information to an artificial intelligence (AI) model; processing the visual information using the AI model to determine a plurality of scales of the excreta; outputting the plurality of scales of the excreta; receiving at least one input to adjust the plurality of scales of the excreta; and providing suggestion based on the adjusted plurality of scales of the excreta.

The obtaining of the visual information of the excreta may comprise at least one of capturing an image; and determining whether there are excreta in the image.

The excreta may be from any one of a baby, an adult, a person, a dog, a cat, and other animals.

The excreta may comprise at least one of urine and stool.

The visual information may be at least one of the video information, image information, three-dimensional information and thermal image information and the visual information may be at least one of diaper visual information, nappy visual information, vessel visual information, litter visual information, and flushing toilet visual information.

If the excreta comprise stool, the plurality of scales of the excreta and the adjusted plurality of scales of the excreta may comprise at least one of a color scale and a consistency scale of the stool.

If the excreta comprise urine, the plurality of scales of the excreta and the adjusted plurality of scales of the excreta may comprise a color scale of the urine.

When outputting the plurality of scales of the excreta, at least one virtual excreta overlaying object may be displayed, and a plurality of scales of the virtual excreta may be displayed according to the determined plurality of scales of the excreta; and/or when the received at least one input is configured to adjust the plurality of scales of the excreta, the plurality of scales of the virtual excreta may be displayed according to the adjusted plurality of scales of the excreta.

If the excreta comprise stool, the plurality of scales of the virtual excreta may comprise at least one of a color scale and a consistency scale of a virtual stool.

If the excreta comprise urine, the plurality of scales of the virtual excreta may comprise a color scale of the virtual urine.

The adjusted plurality of scales of the excreta may be used to re-train the AI model.

The computer-implemented method may further comprise, pre-training the AI model by training visual information, wherein the training visual information may be processed by a loss function, wherein the loss function may comprise mixing two original visual information together according to different transparency, generating mixing coefficients randomly for a plurality of times and letting the AI model learn according to a data distribution during the pre-training.

Before processing the visual information using the AI model, the visual information may be pre-processed by comprising at least one at least one of flipping, lightening, darkening, and cropping.

RandAugment may be used to perform automated data augmentation to the training visual information and/or a FixMatch semi-supervised training method may be used to train the AI model.

The suggestion may comprise at least one health suggestion, which may comprise at least one of diet nutrient recommendation to improve stool function and health, at least one immunity system indicator of the and nutrition guidance to a breastfeeding mother.

The suggestion may comprise at least one meal plan, which may be further based on the dietary status and at least one of gender, age, disease record, weight, and body length, the dietary status being at least one of diet preference and allergen information.

The suggestion may comprise comparable data of cases which have same scales of the excreta as the adjusted plurality of scales of the excreta.

The suggestion may comprise a gut health score calculated based on the adjusted plurality of scales of the excreta.

The suggestion may be provided via a suggestion AI model.

If the adjusted color scale indicates stool with a red, maroon, black, or pale color, an alert may be displayed, and/or the suggestion may be further based on diet record by checking whether the diet record contains cause of the red, maroon, black, or pale color.

The determining of the suggestion is further based on at least one of recorded diet information and at least one of gender, age, disease record, weight, and body length.

An apparatus may be configured to perform the above method.

A storage medium may store instructions, wherein the instructions may be configured to cause a processor to perform the above method.

1. A computer-implemented method of analyzing excreta, comprising:
obtaining visual information of excreta;
providing the visual information to an artificial intelligence (AI) model;
processing the visual information using the AI model to determine a plurality of scales of the excreta;
outputting the plurality of scales of the excreta;
receiving at least one input to adjust the plurality of scales of the excreta; and
providing suggestion based on the adjusted plurality of scales of the excreta.

2. The computer-implemented method according to claim 1, wherein the obtaining of the visual information of the excreta comprises at least one of
capturing an image; and
determining whether there are excreta in the image.

3. The computer-implemented method according to claim 1, wherein the excreta are from any one of a baby, an adult, a person, a dog, a cat, and other animals.

4. The computer-implemented method according to any of the preceding claims, wherein the excreta comprise at least one of urine and stool.

5. The computer-implemented method according to any of the preceding claims, wherein the visual information is at least one of the video information, image information, three-dimensional information and thermal image information and the visual information is at least one of diaper visual information, nappy visual information, vessel visual information, litter visual information, and flushing toilet visual information.

6. The computer-implemented method according to any of the preceding claims, wherein if the excreta comprise stool, the plurality of scales of the excreta and the adjusted plurality of scales of the excreta comprise at least one of a color scale and a consistency scale of the stool.

7. The computer-implemented method according to any of the preceding claims, wherein if the excreta comprise urine, the plurality of scales of the excreta and the adjusted plurality of scales of the excreta comprise a color scale of the urine.

8. The computer-implemented method according to any of the preceding claims, wherein when outputting the plurality of scales of the excreta, at least one virtual excreta overlaying object is displayed, and a plurality of scales of the virtual excreta are displayed according to the determined plurality of scales of the excreta; and/or
wherein when the received at least one input is configured to adjust the plurality of scales of the excreta, the plurality of scales of the virtual excreta are displayed according to the adjusted plurality of scales of the excreta.

9. The computer-implemented method according to claim 8, wherein if the excreta comprise stool, the plurality of scales of the virtual excreta comprise at least one of a color scale and a consistency scale of a virtual stool.

10. The computer-implemented method according to claims 8 and 9, wherein if the excreta comprise urine, the plurality of scales of the virtual excreta comprises a color scale of the virtual urine.

11. The computer-implemented method according to any of the preceding claims, wherein the adjusted plurality of scales of the excreta are used to re-train the AI model.

12. The computer-implemented method according to any of the preceding claims, further comprising, pre-training the AI model by training visual information, wherein the training visual information is processed by a loss function, wherein the loss function comprises mixing two original visual information together according to different transparency, generating mixing coefficients randomly for a plurality of times and letting the AI model learn according to a data distribution during the pre-training.

13. The computer-implemented method according to any of the preceding claims, wherein before processing the visual information using the AI model, the visual information is pre-processed by comprising at least one at least one of flipping, lightening, darkening, and cropping.

14. The computer-implemented method according to any of claims 12 and 13, wherein RandAugment is used to perform automated data augmentation to the training visual information and/or a FixMatch semi-supervised training method is used to train the AI model.

15. The computer-implemented method according to any of the preceding claims, wherein the suggestion comprises at least one health suggestion, which comprises at least one of diet nutrient recommendation to improve stool function and health, at least one immunity system indicator of the and nutrition guidance to a breastfeeding mother.

16. The computer-implemented method according to any of the preceding claims, wherein the suggestion comprises at least one meal plan, which is further based on the dietary status and at least one of gender, age, disease record, weight, and body length, the dietary status being at least one of diet preference and allergen information.

17. The computer-implemented method according to any of the preceding claims, wherein the suggestion comprises comparable data of cases which have same scales of the excreta as the adjusted plurality of scales of the excreta.

18. The computer-implemented method according to any of the preceding claims, wherein the suggestion comprises a gut health score calculated based on the adjusted plurality of scales of the excreta.

19. The computer-implemented method according to any of the preceding claims, wherein the suggestion is provided via a suggestion AI model.

20. The computer-implemented method according to any of the preceding claims, wherein if the adjusted color scale indicates stool with a red, maroon, black, or pale color, an alert is displayed, and/or the suggestion is further based on diet record by checking whether the diet record contains cause of the red, maroon, black, or pale color.

21. The computer-implemented method according to any of the preceding claims, wherein the determining of the suggestion is further based on at least one of recorded diet information and at least one of gender, age, disease record, weight, and body length.

22. An apparatus configured to perform the method in any of the preceding claims.

23. A storage medium storing instructions, wherein the instructions are configured to cause a processor to perform any of the claims 1 to 21.
